(19) United States
(12) Patent Application Publication
(10) Pub. No.: US 2009/0221983 A1
(43) Pub. Date: Sep. 3, 2009

(54) Thong style urinary pad garment

(76) Inventor: Richard W. Schanz, St. George, UT (US)

Correspondence Address: MARCUS G THEODORE, PC, 466 SOUTH 500 EAST, SALT LAKE CITY, UT, 84102, US

(21) Appl. No.: 12/387,503

(22) Filed: May 4, 2009

Related U.S. Application Data
(63) Continuation-in-part of application No. 11/807,090, filed on May 25, 2007, now abandoned.
(60) Provisional application No. 60/843,553, filed on Sep. 11, 2006.

(51) Int. Cl. A61F 13/15

(52) U.S. Cl. 604/385.15; 604/394

(57) ABSTRACT

A women's reusable, decorative thong garment, with a pouch to hold interchangeable urinary pads structured to provide the wearer with a feeling of security that the pad will not be exposed or fallout.

### RELATED APPLICATIONS

This application is a continuation-in-part of the patent application entitled “Thong Style Urinary Pad Garment”, Ser. No. 11/807,090 filed May 25, 2007, which is based on provisional patent application entitled “Thong Style Urinary Pad Garment” filed Sep. 11, 2006 and assigned Ser. No. 60/843,553.

### BACKGROUND OF THE INVENTION

1. Field of Invention

The present invention relates to garments to hold bladder leakage pads. In particular, it relates to a women's thong style garment with a pouch to hold replaceable urinary pads.

2. Description of Related Art

Various sanitary napkins and urinary pad holders are known in the art. Kramer, U.S. Des. Pat. No. 445,181 issued Jul. 17, 2001 provides an absorbent panty liner for a thong. Coleman, U.S. Pub. No. 2005/0090795 published Apr. 28, 2005 discloses a disposable sanitary panty containing a thickened- crotch pad held by a bikini type panty.

Clyne, U.S. Pat. No. 2,977,957 issued Aug. 8, 1957 discloses a sanitary napkin holder.

Lee, U.S. Pub. No. 2005/0267438 published Dec. 1, 2005 discloses a disposable thong under pant, which may be combined with a full sanitary napkin. Leathers, U.S. Pat. No. 4,940,463 issued Jul. 10, 1990 discloses another bikini disposable combined panty with sanitary napkin.

Darby, U.S. Pat. No. 5,683,373 issued Nov. 4, 1997 discloses a sanitary napkin shaped for use with a thong garment.

Gell et al, U.S. Pat. No. 6,997,915 issued Feb. 14, 2006 discloses a sanitary napkin with adjustable length intergluteal strip Bell et al, U.S. Pub No. 2004/0102747 published May 27, 2004 discloses a liner for use in combination with thong, G-string, tanga or T-back underwear. Darby, U.S. Des. Pat. No. 402,756 issued Dec. 15, 1998 discloses a garment shield.

Cited for general interest is the Oram, U.S. Pat. No. 5,388,275 issued Feb. 14, 1995 disclosing protective wear for female water skiers.

None of the above references provides a reusable, washable, thong style decorative garment with a pouch to hold a replaceable urinary pad for women with intermittent bladder leakage.

### SUMMARY OF THE INVENTION

The present invention comprises a women's thong style urinary pad holder. It is a washable, reusable garment for women styled like a thong as shown in the attached drawings. The thong has a low V or T cut front with side and bottom ends and a T or V shaped back with side and bottom ends whose side ends when connected form a waistband and are structured to removably secure around the waist. The bottoms ends of the front and back are connected to a crotch pouch positioned to cover the pubic area. The thong style urinary pad holder is constructed of a washable fabric of silk, sateen, lace, satin, nylon, cotton, and/or washable leather. The front of the garment is made of a material, which may be plain or decorated with designs, sayings, sequins, and/or rhinestones.

To make it easy for the wearer to put the thong on or take it off, preferably at least one of the side ends of the waistband has openable fasteners, such as hook and loop strips, snaps, etc. In one preferred embodiment, both sides of the waistband have openable fasteners. Alternatively, the waistband side ends may be elasticized, or have elastic panels on each side in the waistband. In one preferred embodiment, the waistband has two to three segments of elastic or elasticized material for proper fit or to accommodate different sized wearers.

In the crotch area a pouch is sewn to the bottom ends of the front and back such that it is positioned proximate the urethra of a wearer, when worn. The pouch has an opening and is structured to accommodate a replaceable urinary pad removably inserted into the pouch to absorb occasional or intermittent leakage of the bladder caused by anxiety, nervousness, or too long between rest stops, age, or a medical problem. The pouch is preferably constructed to be of a compatible or contrasting fabric or color as the thong. The preferred dimensions of the pouch is approximating four inches wide by ten inches long to accommodate a standardized urinary pad. Preferred urinary pads have the side of the urinary pad next to the user constructed of a material, which allows fluids to pass into the urinary pad for absorption. The opposite exterior of the pad may be constructed of a fluid impervious material to help minimize accidents.

Elastic is sewn around the edges of the pouch open end so that when the urinal pad is placed in the pouch, it is held therein.

The invention thus provides a reusable, decorative thong garment, with a pouch to hold interchangeable urinary pads and structured to provide the wearer with a feeling of security that the pad will not be exposed or fall out.

### BRIEF DESCRIPTION OF THE DRAWINGS

FIG. 1 is a bottom view of a preferred embodiment of the invention.

FIG. 2 is a front view of the embodiment shown in FIG. 1.

FIG. 3 is a back view of the embodiment shown in FIG. 1

FIG. 4 is another preferred back view of a back

### DESCRIPTION OF THE ILLUSTRATED EMBODIMENTS

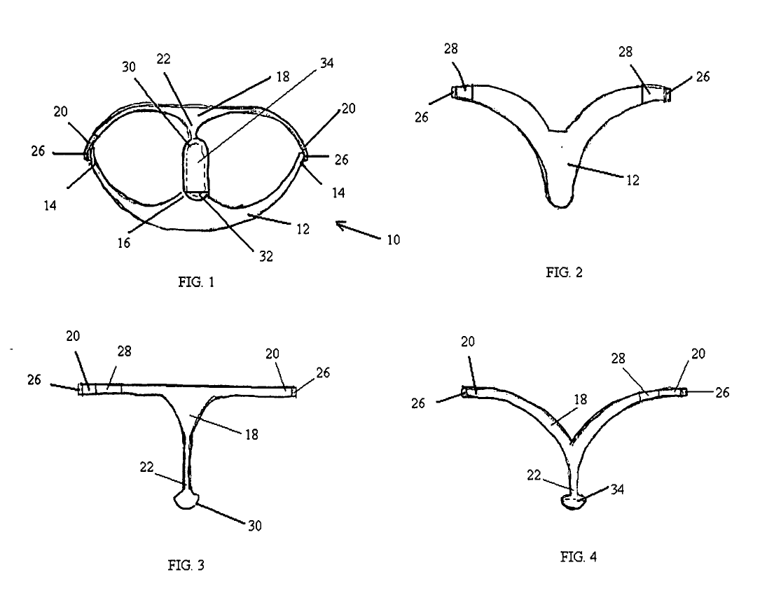

FIG. 1 illustrates a bottom view of a preferred embodiment of the invention, which comprises a washable, reusable thong garment 10 structured to removably hold urinary pads proximate the urethra of women. The thong garment 10 has a low V cut front 12 with side ends 14 and a bottom end 16 and a T or V shaped back 18 with side ends 20 and a bottom end 22 whose side ends 20 when connected to the front side ends 14 form a waistband structured to removably secure around the waist. The thong garment 10 is constructed of a washable fabric of silk, sateen, lace, satin, nylon, cotton, and/or washable leather. The front 12 of the thong garment can be plain or decorated with designs, sayings, sequins, and/or rhinestones.

To make it easy for the wearer to put the thong garment 10 on or take it off, each side ends 14, 22 in the waistband belt area has a Velcro fastener 26. On each side of the Velcro fastener 26 (or other fastener) are two to three inches of elastic or elasticized material 28, which stretch and contract for proper fit or to accommodate different sized wearers. In the crotch area, a pouch 30 is sewn as shown to hold and position a urinary pad 34 therein to absorb occasional or-intermittent leakage of the bladder.

The pouch 30 is to be of a compatible or contrasting fabric or color approximating four inches wide by ten inches long to accommodate a standardized urinary pad. Elastic is to be sewn around the edges of the pouch 30 opening 32 so that when a urinary pad 34 is placed in the pouch 30, it is held in by tension giving the wearer a feeling of security that the urinary pad 34 will not be exposed or fallout.

FIG. 2 is a front view of the embodiment shown in FIG. 1 showing 1 plunging front 12, which is decorated or patterned to suit the preference of a user.

FIG. 3 is a back view of the embodiment shown in FIG. 1 with a T shaped back 18.

FIG. 4 is back view of a V shaped back 18 with a plunging waistband.

Either the straight back or plunging waistband style is employed, depending upon the user's preferences.

The invention thus provides a reusable, decorative thong garment 10, with a pouch 30 to hold interchangeable urinary pads 34 and structured to provide the wearer with a feeling of security that the pad will not be exposed or fall out.

The above description and specification should not be construed as limiting the scope of the claims. The claims themselves recite those features deemed essential to the invention.

I claim:

1. A women's thong style urinary pad holder comprising:
a. a V or T shaped front with side ends and a bottom end.
b. a V or T shaped back with side ends and a bottom end, the back side ends and the front side ends removably connected to form a waistband to removably secure around the waist,
c. a crotch pouch with an opening structured to accommodate a replaceable urinal pad removably inserted into the crotch pouch attached to the front and back bottom ends to support and position the crotch pouch proximate the urethra of a wearer.

2. A women's thong style urinary pad holder according to claim 1, wherein the thong style urinary pad holder is constructed of a washable fabric selected from the group consisting of silk, sateen, lace, satin, nylon, cotton, and/or washable leather.

3. A women's thong style urinary pad holder according to claim 1, wherein the front of the garment is made of a material, which may be plain or decorated with designs, sayings, sequins, and/or rhinestones.

4. A women's thong style urinary pad holder according to claim 1, wherein at least one of the side ends has openable fasteners.

5. A women's thong style urinary pad holder according to claim 1, wherein the side ends are made of elastic or elasticized material for proper fit or to accommodate different sized wearers.

6. A women's thong style urinary pad holder according to claim 1, wherein the pouch is constructed to be of a compatible or contrasting fabric or color as the thong.

7. A women's thong style urinary pad holder according to claim 1 wherein the dimensions of the crotch pouch is approximating four inches wide by ten inches long.

8. A women's thong style urinary pad holder according to claim 1, wherein the edges of the crotch pouch opening are elasticized and expandable so that when a urinal pad is placed in the pouch, it is held therein under tension.

9. A women's thong style urinary pad holder according to claim 1, wherein the women's thong style urinary pad holder is constructed of a washable, reusable fabric.

10. A women's thong style urinary pad holder comprising:
a. a washable decorative V or T shaped front with elasticized side ends sized to extend around the front of the waist, at least one of the front side ends adapted with an openable fasteners and a bottom end,
b. a washable V or T shaped back with elasticized side ends sized to extend around the back of the waist, and a bottom end, at least one of the back side ends having corresponding fasteners to removably secure the front and back to form a waistband around the waist, and the back side ends,
c. a washable crotch pouch with an elasticized expandable opening structured to accommodate and hold in the pouch under tension a replaceable urinary pad removably inserted into the crotch pouch attached to the front and back bottom ends to support and position the crotch pouch proximate the urethra of a wearer.
